(19) United States
(12) Patent Application Publication
(10) Pub. No.: US 2004/0047835 A1
(43) Pub. Date: Mar. 11, 2004

(54) Combinatorial drug therapy using polymer drug conjugates

(75) Inventor: James A. Bianco, Seattle, WA (US)

Correspondence Address: DONALD W. WYATT, CELL THERAPEUTICS, INC., 501 ELLIOTT AVENUE WEST, #400, SEATTLE, WA, 98119, US

(73) Assignee: Cell Therapeutics, Inc.

(21) Appl. No.: 10/635,970

(22) Filed: Aug. 6, 2003

Related U.S. Application Data
(63) Continuation of application No. 60/408,591, filed on Sep. 6, 2002.
(60) Provisional application No. 60/409,159, filed on Sep. 9, 2002.
(60) Provisional application No. 60/419,512, filed on Oct. 18, 2002.

(51) Int. Cl. A61K 38/16; A61K 38/14; A61K 31/7072; A61K 31/4745

(52) U.S. Cl. 424/78.17; 514/8; 514/283; 514/109; 514/50; 514/34; 600/1

(57) ABSTRACT

The present invention discloses combinations of drug conjugates with other therapeutic agents, including chemotherapy drugs. The invention also provides methods of using the combinations for the treatment of diseases associated with cell proliferation, such as tumors.

### CROSS-REFERENCE TO RELATED PATENT APPLICATIONS

This application claims the benefit of US Provisional Applications Serials No. 60/408,591 filed Sep. 6, 2002, No. 60/409,159 filed Sep. 9, 2002 and 60/419,512 filed Oct. 18, 2002, all herein incorporated by reference.

### FIELD OF THE INVENTION

The present invention relates generally to the fields of pharmaceutical compositions to be used in the treatment of cancer. The present invention also relates to the field of pharmaceutical preparations of anticancer agents such as taxanes and camptothecins.

### BACKGROUND OF THE INVENTION

Given the large number of different molecular defects and pathologies associated with tumor formation and progression, it is not surprising that such a wide range of chemotherapeutic agents, targeting a variety of different cellular pathways, have been identified. Chemotherapeutic drugs may be classified into a large number of groups, based upon their mechanism of action, including, for example, platinates, alkylating agents, antimetabolites, plant alkaloids, antimicrotubule agents, antibiotics, hormonal agents, interleukins, mitotic inhibitors, angiogenesis inhibitors, apoptosis promoters, and biological response modifiers. Typically, each of these classes of drugs acts to inhibit tumor cell growth or proliferation via a different molecular mechanism. For example, selective estrogen-receptor modulators bind to estrogen receptors of estrogen-dependent breast cancer cells and prevent estrogen binding, thereby effectively starving these cancer cells. In completely different modes of action, nucleoside analogs, such as azacytidine and flurouracil, inhibit nucleic acid synthesis and metabolism.

The taxanes are an important class of chemotherapeutic drugs that act as mitotic spindle inhibitors and enhance tubulin polymerization, thereby inhibiting cancer cell division. Commercially available taxanes used for the treatment of a variety of cancers include paclitaxel (TAXOL®) and docetaxel (TAXOTERE®).

Paclitaxel, an anti-microtubule agent extracted from the needles and bark of the Pacific yew tree, Taxus brevifolia, has shown a remarkable anti-neoplastic effect in human cancer in clinical trials. This has been reported primarily in advanced ovarian and breast cancer, although taxanes are increasingly being evaluated and used to treat other cancers. For example, significant activity has been documented in small-cell and non-small cell lung cancer, head and neck cancers, and in metastatic melanoma.

In December 1992, the U.S. Food and Drug Administration (FDA) approved the use of paclitaxel for ovarian cancer that was resistant to treatment (refractory). Paclitaxel was later approved as initial treatment for ovarian cancer in combination with cisplatin. Women with epithelial ovarian cancer now generally treated with surgery followed by a taxane and a platinum. The FDA has also approved paclitaxel for the treatment of breast cancer that recurred within 6 months after adjuvant chemotherapy (chemotherapy that is given after the primary treatment to enhance the effectiveness of the primary treatment), or that spread (metastasized) to nearby lymph nodes or other parts of the body. Paclitaxel is also used for other cancers, including AIDS-related Kaposi's sarcoma and lung cancer. However, a major difficulty in the development of paclitaxel for clinical use has been its insolubility in water.

Docetaxel is semisynthetically produced from 10-deacetyl baccatin III, a noncytotoxic precursor extracted from the needles of Taxus baccata and esterified with a chemically synthesized side chain (Cortes and Pazdur, Journal of Clinical Oncology 13:2643-2655 (1995)). Various cancer cell lines, including breast, lung, ovarian, and colorectal cancers and melanomas have been shown to be responsive to docetaxel. In clinical trials, docetaxel has been used to achieve complete or partial responses in breast, ovarian, head and neck cancers, and malignant melanoma.

Paclitaxel is typically formulated as a concentrated solution containing paclitaxel 6 mg per milliliter of Cremophor EL (polyoxyethylated castor oil) and dehydrated alcohol (50% v/v) and must be further diluted before administration (Goldspiel, Ann. Pharmacotherapy 28:S23-26 (1994)). The amount of Cremophor EL necessary to deliver the required doses of paclitaxel is significantly higher than that administered with any other drug that is formulated in Cremophor. Several toxic effects have been attributed to Cremophor, including vasodilation, dyspnea, and hypotension. This vehicle has also been shown to cause serious hypersensitivity in laboratory animals and humans. In fact, the maximum dose of paclitaxel that can be administered to mice by i.v. bolus injection is dictated by the acute lethal toxicity of the Cremophor vehicle. In addition, Cremophor EL, a surfactant, is known to leach phthalate plasticizers such as di(2-ethylhexyl)phthalate (DEHP) from the polyvinylchloride bags and intravenous administration tubing. DEHP is known to cause hepatotoxicity in animals and is carcinogenic in rodents. This preparation of paclitaxel is also shown to form particulate matter over time and thus filtration is necessary during administration. Therefore, special provisions are necessary for the preparation and administration of paclitaxel solutions to ensure safe drug delivery to patients, and these provisions inevitably lead to higher costs.

Recently, significant advancements have been made in the development of water-soluble formulations of paclitaxel. The anticancer agent poly(L-glutamic acid)-paclitaxel (PGTXL), a conjugate of paclitaxel and the water-soluble polyglutamate carrier, has been shown to possess superior anti-tumor activity as compared to free paclitaxel. Analysis of the pharmacological action of PGL-TXL revealed that paclitaxel is released from the PG-TXL conjugate in vitro, and the released paclitaxel is subsequently transported into cells, where it disturbs microtubule polymerization. Additional studies demonstrated that both PG-TXL and free paclitaxel induced a G2/M arrest in the cell cycle. These results suggest that PG-TXL exerts its antitumor activity by continuous release of free paclitaxel. Water soluble paclitaxel conjugates and methods of producing the same are described in U.S. Pat. No. 5,977,163 and U.S. Pat. No. 6,262,107, which are hereby incorporated by reference in their entirety.

The camptothecin are another plant akyloid useful for treating human malignancies. However, the therapeutic efficacy of 20(s)-camptothecin (CPT) is limited in humans by the instability of the active lactone form due to preferential binding of the carboxylate to serum albumin and by difficulty in formulation. The linkage of 20(s)-camptothecin to poly-(L-glutamic acid) enhanced solubility and improved distribution to tumors through enhanced permeability and retention. Furthermore, in athymic mice bearing ectopic human colon or lung tumors, the efficacy of CPT linked to polyglutamate was enhanced compared to free camptothecin. Ibid. Thus, conjugation of CPT to polyglutamate enhanced pharmaceutical properties and preclinical efficacy.

Since different classes of chemotherapeutic agents generally have different mechanisms of action, combinations of chemotherapeutic agents are often used in an effort to attack tumor cells through multiple mechanisms and thereby more effectively halt tumor growth and kill tumor cells. Combination therapy using multiple classes of chemotherapeutic agents is also used to avoid cross-resistance to drugs. Taxanes have been used in combination with a variety of other antitumor drugs, including, for example, the cyclin-dependent kinase inhibitor, flavopiridol, the platinum-based drug, carboplatin, the peptidomimetic inhibitor of farnesyl transferase, ER-51785, the EGFR-selective tyrosine kinase inhibitor, IRESSA (gefitinib, ZD1839), cyclosporine, and trastuzumab.

Combination drug therapy is frequently limited due to toxic side effects associated with the combination of drugs. These undesirable side effects may be associated with either the drug or its delivery vehicle. For example, the use of paclitaxel in combination with other chemotherapeutic agents is limited by the acute lethal toxicity of the Cremophor vehicle and paclitaxel-associated neutropenia. Such side effects pose particular problems for combination therapy when the drugs cause similar side effects or contain the same toxic vehicle. In such circumstances, it is frequently not possible to administer each drug at the dosage found to be the most effective when used alone. Accordingly, suboptimal doses may be used, with the effectiveness of each drug compromised.

Accordingly, there is a need in the art for improved combinations of drugs, including taxanes and other chemotherapeutic agents, new compositions allowing the delivery of increased doses of combination chemotherapeutic agents to patients in need thereof, and improved methods of treating cancer using combined chemotherapy. The present invention fulfills these needs and further provides other related advantages.

### BRIEF SUMMARY OF THE INVENTION

### DETAILED DESCRIPTION OF THE INVENTION

All references to patents, patent applications and publications are herein incorporated by reference.

The present invention provides compositions comprising a drug conjugated to a polymer or derivatized with a chelating agent. The compositions of the present invention provide distinct advantages over existing compositions for combination drug therapy, particularly for the treatment of cancer. For example, the compositions of the invention exhibit reduced toxicity and side effects, thereby allowing combination treatment with higher doses or for longer time periods as compared to treatment using non-conjugated or non-derivatized drugs. Accordingly, any one or more of the therapeutic agents used in combination drug therapy may be used at a higher dose, for an increased duration or longer time period, or be administered more frequently.

Conjugation of chemotherapeutic drugs to polymers is an attractive approach to reduce systemic toxicity and improve the therapeutic index. Polymers with molecular mass larger than 30 kDa do not readily diffuse through normal capillaries and glomerular endothelium, thus sparing normal tissue from irrelevant drug-mediated toxicity (Maeda and Matsumura, 1989; Reynolds, 1995). On the other hand, it is well established that malignant tumors often have disordered capillary endothelium and greater permeability than normal tissue vasculature. Thus, a polymer-drug conjugate that would normally remain in the vasculature may selectively leak from blood vessels into tumors, resulting in tumor accumulation of active therapeutic drug. Additionally, polymer-drug conjugates may act as drug depots for sustained release, producing prolonged drug exposure to tumor cells. Finally, water soluble polymers may be used to stabilize drugs, as well as to solubilize otherwise insoluble compounds.

In one embodiment, a composition of the invention comprises a chemotherapeutic agent, such as a taxane or camptothecin, conjugated to a polymer, such as poly-(L-glutamic acid). The poly-(L-glutamic acid)-paclitaxel (PG-TXL) conjugate displayed increased efficacy and decreased toxicity when administered to tumor-bearing hosts as compared with the unconjugated form of paclitaxel. U.S. Pat. No. 5,977,163 and U.S. Pat. No. 6,262,107. PG-TXL also exhibited increased water solubility, a slower clearance from the body, and an increased accumulation in the tumor. PG-TXL is the prototypic example used to exemplify the invention throughout the specification, but it should be understood that other drug conjugates are also included within the invention.

In one embodiment, PG-TXL is used in combination with a chemotherapeutic agent, such as gemcitabine. Previous attempts to combine paclitaxel and gemcitidine have been dose-limited, due to the fact that both paclitaxel and gemcitabine are associated with neutropenia. In fact, the dose limiting toxicities of the combination of gemcitabine and paclitaxel and the combination of gemcitabine, carboplatin, and paclitaxel were both neutropenia.

The novel combinations and methods of the present invention provide significant advances over prior methods and compositions. For example, conjugated paclitaxels improve the efficacy of paclitaxel-based anti-cancer therapy by providing water-soluble and controlled-release paclitaxel-derived compositions. Such compositions also reduce or eliminate the need for solvents, such as Cremophor, that are associated with side effects seen with prior paclitaxel compositions. In addition, the combinations and methods of the present invention permit using increased doses of one or more chemotherapeutic agents or treating a patient for a longer duration or over longer time periods.

A. Compositions

According to the methods of the invention, combination therapy may be performed using any drug conjugate of the invention. Drug conjugates of the invention include drugs conjugated to a polymer and drugs conjugated or derivatized with a chelating agent. In certain embodiments, conjugation or derivatization of the drug causes the drug to be more water soluble, more readily taken up by a cell or tissue, or less toxic. Furthermore, in specific embodiments, conjugation or derivatization allows the conjugated drug to be administered at a higher equivalent dose, more frequently, or with less associated toxic side effects than the free drug. In another specific embodiment of the invention, conjugation of the drug allows the conjugated to be administered at a lower equivalent dose or less frequently with similar or greater efficacy than the free drug. Specific polymers, chelating agents, and conjugated drugs that may be used according to the invention are described below

1. Conjugates

Drug conjugates of the invention include any known or discovered drug conjugated to any polymer. Drug conjugates may be used alone or in combination with other drugs to treat a variety of diseases, including, for example, diseases associated with undesirable cell proliferation, such as cancer, restenosis, and inflammatory diseases. In certain embodiments of the invention, the conjugated drugs must be capable of being directly conjugated to a polymer, while in other embodiments, the drug may be conjugated via an intermediary molecule, such as a linker molecule. Drug conjugates of the invention typically exhibit one or more desired or beneficial properties, such as increased water solubility, reduced toxicity, increased stability, and increased absorption, for example. In addition, drug conjugates, or at least the polymer portion of the conjugate, may be substantially non-antigenic. The term “substantially non-antigenic” refers to a substance that does not substantially bind specifically or selectively to an antibody or a T-cell receptor, under appropriate conditions. For example, a substantially non-antigenic substance is one that does not elicit a statistically significant antigenic response from a vertebrate as detected by ELISA assay as compared to a positive control.

a. Polymers

Polymers conjugated to drugs according to the invention include all known and discovered polymers. Examples of polymers are described in U.S. Pat. No. 5,977,163 and U.S. Pat. No. 6,262,107, which are hereby incorporated by reference in their entirety. In certain embodiments, a polymer possesses physiochemical qualities including being non-immunogenic, non-allergenic, or non-antigenic, being metabolizable, being large molecular weight, being soluble, particularly in aqueous physiological solutions such as phosphate buffered saline, for example, and capable of being conjugated (e.g., covalently bound) or associated (e.g., admixed with or associated through charge-charge interactions) with a drug.

The term “poly (amino acid) polymer”, as used herein, refers to a polymer comprised of naturally occurring or synthetic amino acids either as a heteropolymer or homopolymer. The amino acids need not be polymerized through peptide bonds but may be bound in any fashion that allows amino acid monomers to be bound sequentially.

The term “poly (anionic amino acid) polymer”, as used herein, refers to a polymer comprised of amino acid monomers such that the polymer exhibits a net anionic character.

The terms “poly-glutamic acid” or “poly-glutamic acids” include poly (1-glutamic acid), poly (d-glutamic acid) and poly (dl-glutamic acid), the terms “a poly-aspartic acid” or “polyaspartic acids” include poly (l-aspartic acid), poly (d-aspartic acid), and poly (dl-aspartic acid), the terms “a poly-lysine” or “poly-lysines” include poly (l-lysine), poly (d-lysine), and poly (dl-lysine), the terms “a poly-serine” or “poly-serines” include poly (l-serine), poly (d-serine), and poly (dl-serine), the terms “a poly-glycine” or “poly-glycines” include poly (l-glycine), poly (d-glycine), and poly (dl-glycine), the terms “a poly-alanine” or “poly-alanines” include poly (1-alanine), poly (d-alanine), and poly (dl-alanine), and the terms “a poly-cysteine” or “poly-cysteines” include poly (l-cysteine), poly (d-cysteine), and poly (dl-cysteine). The terms “a water soluble polyamino acid”, “water soluble polyamino acids”, or “water soluble polymer of amino acids” include, but are not limited to, poly-glutamic acid, poly-aspartic acid, poly-lysine, and amino acid chains comprising mixtures of glutamic acid, aspartic acid, and/or lysine.

In certain embodiments, the terms “a water soluble polyamino acid,” “water soluble polyamino acids,” or “water soluble polymer of amino acids” include amino acid chains comprising combinations of glutamic acid and/or aspartic acid and/or lysine, of either d and/or l isomer conformation. In certain embodiments, such a “water soluble polyamino acid” contains one or more glutamic acid, aspartic acid, and/or lysine residues.

In certain aspects, the carrier is a polymer, which may be synthetic or natural. Further, the polymer carrier may be substantially non-antigenic or biodegradable, or both. In certain embodiments, the compositions of the present invention may comprise a wide variety of polymers. In one embodiment, the polymers can be a poly(diene), a poly(alkene), a poly(acrylic), a poly(methacrylic), a poly(vinyl ether), a poly(vinyl alcohol), a poly(vinyl ketone), a poly(vinyl halide), a poly(vinyl nitrile), a poly(vinyl ester), a poly(styrene), a poly(carbonate), a poly(ester), a poly(orthoester), a poly(esteramide), a poly(anhydride), a poly(urethane), a poly(amide), a cellulose ether, a cellulose ester, a poly(saccharide), poly(lactide-co-glycolide), a poly(lactide), a poly(glycolide), a copolyoxalate, a polycaprolactone, a poly(lactide-co-caprolactone), a poly(esteramide), a polyorthoester, a poly(a-hydroxybutyric acid), a polyanhydride or a mixture thereof. In particular embodiments, the polymers comprise a poly(lactide-co-glycolide), a poly(lactide), a poly(glycolide), such as polyethylene glycol (PEG), a copolyoxalate, a polycaprolactone, a poly(lactide-co-caprolactone), a poly(esteramide), a polyorthoester, a poly(a-hydroxybutyric acid), a polyanhydride, or a mixture thereof.

Polymers may also be polymers derived from the polymerization of at least one monomer. Thus, in another embodiment, the polymers may be a polymer or oligomer derived from the polymerization or oligomerization of at least one monomer. Examples of suitable monomers include an alpha hydroxycarboxylic acid, a lactone, a diene, an alkene, an acrylate, a methacrylate, a vinyl ether, a vinyl alcohol, a vinyl ketone, a vinyl halide, a vinyl nitrile, a vinyl ester, styrene, a carbonate, an ester, an orthoester, an esteramide, an anhydride, a urethane, an amide, a cellulose ether, a cellulose ester, a saccharide, an alpha hydroxycarboxylic acid, a lactone, an esteramide, or a mixture thereof.

In other embodiments, the polymers are the polymerization products of an alpha hydroxycarboxylic acid, a lactone or a mixture thereof. In yet further embodiments, the alpha hydroxycarboxylic acid comprises glycolic acid, lactic acid, a-hydroxy butyric acid, a-hydroxyisobutyric acid, a-hydroxyvaleric acid, a-hydroxyisovaleric acid, a-hydroxy caproic acid, a-hydroxy-a-ethylbutyric acid, a-hydroxyisocaproic acid, a-hydroxy-3-methylvaleric acid, a-hydroxyheptanoic acid, a-hydroxyoctanoic acid, a-hydroxydecanoic acid, a-hydroxymysristic acid, a-hydroxystearic acid, a-hydroxyligoceric acid or a mixture thereof. In one embodiment, the lactone comprises 3-propiolactone, tetramethyleneglycolide, b-butyrolactone, 4-butyrolactone, pivalactone or mixtures thereof.

In certain embodiments, a polymer is derived from one or more amino acids. In other embodiments, the polymers are homopolymers or heteropolymers. In certain embodiments, polymers are amino acids or anionic monomers, such as anionic amino acids, for example. One example of an anionic amino acid for the formation of such polymer carriers is glutamic acid. For example, polyglutamate derived from L-glumatic acid, D-glumatic acid or mixtures, e.g. racemates, of these L and D isomers are used. L and/or D glutanyl, aspartly, glycyl, seryl, threonyl, and cysteinyl are all examples of amino acids that may be used according to the invention.

In other embodiments, the polymers are copolymers, such as block, graft or random copolymers, containing glutamic acid. Thus, copolymers of glutamic acid with at least one other (preferably biodegradable) monomer, oligomer or polymer are included. These include, for example, copolymers containing at least one other amino acid, such as aspartic acid, serine, tyrosine, glycine, ethylene glycol, ethylene oxide, (or an oligomer or polymer of any of these) or polyvinyl alcohol. Glutamic acid may, of course, carry one or more substituents and the polymers include those in which a proportion or all of the glutamic acid monomers are substituted. Substituents include, for example, alkyl, hydroxy alkyl, aryl and arylalkyl, commonly with up to 18 carbon atoms per group, or polyethylene glycol attached by ester linkages. The expression “poly (glutamic acid)” and cognate expressions herein are to be construed as covering any of the aforesaid possibilities unless the context otherwise demands.

In certain embodiments, the polymers are poly(amino acids) including, but not limited to poly(l-glutamic acid), poly(d-glutamic acid), poly(dl-glutamic acid), poly(l-aspartic acid), poly(d-aspartic acid), poly(dl-aspartic acid), poly(l-serine), poly(d-serine), poly(dl-serine), poly(l-tyrosine), poly(d-tyrosine), poly(dl-tyrosine), poly(l-glysine), poly(d-glysine), poly(dl-glysine), poly(l-threonine), poly(d-threonine), poly(dl-threonine), poly(d-cysteine), poly(l-cysteine), and poly(dl-cysteine). In further embodiments, the polymers are copolymers, such as block, graft or random copolymers, of the above listed poly(amino acids) with polyethylene glycol, polycaprolactone, polyglycolic acid and polylactic acid, as well as poly(2-hydroxyethyl 1-glutamine), chitosan, carboxymethyl dextran, hyaluronic acid, human serum albumin and alginic acid, with poly-glutamic acids being particularly preferred.

Polymer carriers of the present invention will generally range from about 1,000 daltons molecular weight to less than 10,000,000 daltons. Although usually not more than about 5,000,000 daltons, polymer carriers of invention have no upper limit to their molecular weight. The polymers of the present invention, in certain embodiments, have a molecular weight of about 10 daltons to about 5,000 daltons, including all integer values within this range, including, for example, 100,200, 300, 500, 1,000, 1,500, 2,000, 2,500,3,000, 3,500, 4,000, and 4,500 daltons, with certain embodiments comprising polymer carriers having a molecular weight of about 600 daltons, about 32,000 daltons or about 33,000 daltons.

In additional embodiments, various substitutions of naturally occurring, unusual, or chemically modified amino acids may comprise the amino acid composition of the poly(amino acid) polymer, and particularly the poly(anionic amino acid) polymers and in certain embodiments the poly-glutamic acid polymers, to produce a poly(amino acid) polymer including, but not limited to polyanionic amino acid polymers having like or otherwise desirable characteristics of a carrier of the present invention. Further, homopolymers of the present invention may comprise polymers that are homo-anionic, for example, comprising strictly anionic amino acids without necessarily being structurally identical.

A poly(amino acid) or poly(anionic amino acid) polymer, such as poly-glutamic acid, poly-aspartic acid, poly-serine, poly-tyrosine, poly-glycine, or water soluble amino acid chain or polymer comprising a mixture of glutamic acid, aspartic acid, serine, tyrosine and/or glycine, may, at the lower end of the amino acid substitution range, have 1, 2, 3, 4, 5, 6, 7, 8, 9, 10, 11, 12, 13, 14, 15, 16, 17, 18, 19, 20, 21, 22, 23, 24, 25 or more glutamic acid, aspartic acid, serine, tyrosine or glycine, residues, respectively, substituted by any of the naturally occurring, modified, or unusual amino acids described herein. In other aspects of the invention, a poly(amino acid) homopolymer such as poly-glutamic acid, poly-aspartic acid, poly-serine, poly-tyrosine, poly-glycine, or a poly(amino acid) copolymer comprising a mixture of some or all of these five amino acids may, at the lower end, have about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, to about 25% or more glutamic acid, aspartic acid, serine, tyrosine or glycine residues, respectively, (% by weight or by residue) and/or substituted by any of the naturally occurring, modified, or unusual amino acids described herein.

A poly(amino acid) homopolymer such as poly-glutamic acid, poly-aspartic acid, poly-serine, poly-tyrosine, or poly-glycine may, at the high end of the amino acid substitution range, has less than 25%, less than 26%, less than 27%, less than 28%, less than 29%, less than 30%, less than 31%, less than 32%, less than 33%, less than 34%, less than 35%, less than 36%, less than 37%, less than 38%, less than 39%, less than 40%, less than 41%, less than 42%, less than 43%, less than 44%, less than 45%, less than 46%, less than 47%, less than 48%, less than 49%, to less than 50% or so of the glutamic acid, aspartic acid, serine, tyrosine, or glycine residues (% by weight or by residue), respectively, substituted by any of the naturally occurring, modified, or unusual amino acids described herein. Preferably, the majority of residues comprise glutamic acid and/or aspartic acid and/or serine and/or tyrosine and/or glycine.

Naturally occurring amino acids for use in the present invention as amino acids or substitutions of a poly(amino acid) are alanine, arginine, asparagine, aspartic acid, citrulline, cysteine, glutamine, glycine, histidine, isoleucine, leucine, lysine, methionine, ornithine, phenylalanine, proline, serine, threonine, tryptophan, tyrosine, valine, hydroxy proline, ε-carboxyglutamate, phenylglycine, or O-phosphoserine.

Non-naturally occurring amino acids for use in the present invention include, for example, β-alanine, α-amino butyric acid, γ-amino butyric acid, γ-(aminophenyl) butyric acid, α-amino isobutyric acid, citrulline, ε-amino caproic acid, 7-amino heptanoic acid, β-aspartic acid, aminobenzoic acid, aminophenyl acetic acid, aminophenyl butyric acid, γ-glutamic acid, ε-lysine, methionine sulfone, norleucine, norvaline, ornithine, d-omithine, p-nitro-phenylalanine, hydroxy proline, 1,2,3,4,-tetrahydroisoquinoline-3-carboxylic acid, and thioproline.

Amino acid substitutions are generally based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents.

To effect more quantitative changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein, and correspondingly a poly(amino acid), is generally understood in the art (Kyte & Doolittle, 1982, incorporated herein by reference). It is known that certain amino acids may be substituted for other amino acids having a similar hydropathic index or score and still retain a similar biological activity. In making changes based upon the hydropathic index, the substitution of amino acids whose hydropathic indices are within +/−2 is preferred, those which are within +/−1 are particularly preferred, and those within +/−0.5 are even more particularly preferred.

It is also understood in the art that the substitution of like amino acids can be made effectively on the basis of hydrophilicity. As detailed in U.S. Pat. No. 4,554,101, the following hydrophilicity values have been assigned to amino acid residues: arginine (+3.0); lysine (+3.0); aspartate (+3.0 +/−1); glutamate (+3.0+/−1); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within +/−2 is preferred, those which are within +/−1 are particularly preferred, and those within +/−0.5 are even more particularly preferred. Hence, in reference to hydrophilicity, arginine, lysine, aspartic acid, and glutamic acid are defined herein as biologically functional equivalents, particularly in water soluble amino acid polymers.

Pseudo-poly(amino acids) may also be used in the present invention. Pseudopoly(amino acids) differ from the poly(amino acids) described above in that dipeptide monomers are covalently bound through other than the normal peptide linkages. Pseudo-poly(amino acids) suitable for use in accordance with the present invention are those, for example in Kohn, J. and Langer, R., Polymerization Reactions Involving the Side Chains of α-L-Amino Acids, J. Amer. Chem. Soc., 109, 917 (1987) and Pulapura, S. and Kohn, J., Biomaterials Based on “Pseudo”-Poly(Amino Acids): A Study of Tyrosine Derived Polyiminocarbonates, J. Polymer Preprints, 31, 23 (1990), each of which are incorporated herein by reference. The pseudo-poly(amino acids) can be used alone or in combination with the mixtures of classical poly(amino acids) and pseudo-poly(amino acids) in accordance with the invention.

The manufacture of a poly(amino acid) polymer is well-known to the person of ordinary skill in the art. For example, a homopolymer of glutamic acid may be prepared in a two-step process, in which (i) glutamic acid is treated with phosgene or an equivalent reagent, e.g. diphosgene, at a temperature of from 15° C. to 70° C. to form an N-carboxyanhydride (NCA), and (ii) ring-opening polymerization of the N-carboxyanhydride is effected with a base to yield poly-(glutamic acid). Suitable bases include alkoxides, e.g. alkali metal alkoxides such as sodium mothoxide, organometallic compounds and primary, secondary or tertiary amines, for example butylamine or triethylamine. See, U.S. Pat. No. 5,470,510. There are numerous known methods for chemically synthesizing poly(amino acids).

In certain aspects, the amino acid polymers of the present invention may be produced recombinantly by any means suitable, such as by utilizing transformed E. coli to produce the same. For example, limited bacterial production of poly (glutamic acid) is described, for example in EP-A-410, 638 (Takeda). Bacterial synthetic processes will commonly yield poly (L-glutamic acid), although bacteria are known that will provide the D-form.

b. Derivatives

Preferred water soluble chelators to be used in the practice of the present invention include, but are not limited to, diethylenetriaminepentaacetic acid (DTPA), ethylenediaminetetraacetic acid (EDTA), 1,4,7,10-tetraazacyclododecane-N,N′,N″,N′″-tetraacetate (DOTA), tetraazacyclotetradecane-N,N′,N″,N″′-tetraacetic acid (TETA), hydroxyethylidene diphosphonate (HEDP), dimercaptosuccinic acid (DMSA), diethylenetriaminetetramethylenephosphonic acid (DTTP) and 1-(p-aminobenzyl)-DTPA, 1,6-diaminohexane-N,N,N′,N′-tetraacetic acid, DPDP, and ethylenebis (oxyethylenenitrilo)tetraacetic acid, with DTPA being the most preferred. A preferred embodiment of the present invention may also be a composition comprising ¹¹¹ In-DTPA-paclitaxel. Chelators are commercially available from fine chemical suppliers, such as Aldrich Chemicals (Milwaukee, Wis.).

In those embodiments in which the paclitaxel or another drug is conjugated to a water soluble metal chelator, the composition may further comprise a chelated metal ion. The chelated metal ion of the present invention may be an ionic form of any one of aluminum, boron, calcium, chromium, cobalt, copper, dysprosium, erbium, europium, gadolinium, gallium, germanium, holmium, indium, iridium, iron, magnesium, manganese, nickel, platinum, rhenium, rubidium, ruthenium, samarium, sodium, technetium, thallium, tin, yttrium or zinc. In certain embodiments, the chelated metal ion will be a radionuclide, i.e. a radioactive isotope of one of the listed metals. Radionuclides include, but are not limited to ⁶⁷Ga, ⁶⁸Ga, ¹¹¹In, ^{99m}Tc, ⁹⁰y, ^{114m}In and ^{193m}Pt.

In certain embodiments of the present invention, DTPA-paclitaxel or other paclitaxel-chelating agent conjugates, such as EDTA-paclitaxel, DTTP-paclitaxel, or DOTA-paclitaxel, for example, may be prepared in the form of water-soluble salts (sodium salt, potassium salt, tetrabutylammonium salt, calcium salt, ferric salt, etc.). These salts will be useful as therapeutic agents for tumor treatment. Secondly, DTPA-paclitaxel or other paclitaxel-chelating agents will be useful as diagnostic agents which, when labeled with radionuclides such as ¹¹¹In or ^{99m}Tc, may be used as radiotracers to detect certain tumors in combination with nuclear imaging techniques. It is understood that in addition to paclitaxel (taxol) and docetaxel (taxotere), other taxane derivatives may be adapted for use in the compositions and methods of the present invention and that all such compositions and methods would be encompassed by the appended claims. Methods of producing such derivatives are described, for example, in U.S. Pat. No. 5,977,163, which is hereby incorporated by reference in its entirety.

c. Drugs

The methods described herein may be used to make polymer or chelator conjugates of a variety of therapeutic agents, contrast agents and drugs, including taxoids, etopside, teniposide, fludarabine, doxorubicin, daunomycin, emodin, 5-fluorouracil, FUDR, estradiol, camptothecin, retinoic acids, verapamil, epothilones and cyclosporin. Indeed, any known or discovered drug may be conjugated to a polymer and used according to the invention. In certain embodiments, agents with a free hydroxyl group are readily conjugated to the polymers by similar chemical reactions as described herein for paclitaxel. Such conjugation would be well within the skill of a routine practitioner of the chemical art, and as such would fall within the scope of the claimed invention. As used herein, conjugated to a polymer means the covalent bonding of the drug to the polymer or chelator.

In certain embodiments, drug conjugated to a polymer or chelating agent are drugs used to treat a disease associated with undesirable or aberrant cell growth or proliferation, such as cancer, restenosis, or inflammatory diseases, for example. Thus, conjugated drugs include chemotherapetic anti-cancer or anti-proliferative drugs and anti-inflammatory drugs.

In one embodiment, a taxoid or taxane is conjugated to a polymer or chelating agent. A “taxoid” is understood to mean those compounds that include paclitaxels and docetaxel, and other chemicals that have the taxane skeleton (Cortes and Pazdur, 1995). Taxoids may be isolated from natural sources such as the Yew tree or from cultured cells, or taxoids may be chemically synthesized molecules. In one embodiment, a taxoid is a chemical of the general chemical formula, C₄₇H₅₁NO₁₄, including [2aR-[2aα, 4β, 4α, β, 6β, 9α(αR*,βS*), 11α, 12α, 12aα, 12bα,]]-β-(Benzoylamino)-α-hydroxybenzenepropanoic acid 6, 12b, bis(acetyloxy)-12-(benzoyloxy)2a,3,4,4a,5,6,9,10,11,12,12a, 12b-dodeca hydro-4, 11-dihydroxy-4a,8, 13,13-tetramethyl-5-oxo-7, 11-methano-1H-cyclodeca [3,4]benz-[1,2-b]oxet-9-yl ester. It is understood that paclitaxel and docetaxel are each more effective than the other against certain types of tumors, and that in the practice of the present invention, those tumors that are more susceptible to a particular taxoid would preferably, but not necessarily, be treated with that water soluble taxoid conjugate. Examples of taxane compounds and methods for their preparation are set forth in U.S. Pat. No. 4,942,184.

In another embodiment, a camptothecin is conjugated to a polymer or chelator.

Camptothecin (CPT) compounds include various 20(S)-camptothecins, analogs of 20(S)camptothecin, and derivatives of 20(S)-camptothecin. Camptothecin, when used in the context of this invention, includes the plant alkaloid 20(S)-camptothecin, both substituted and unsubstituted camptothecins, and analogs thereof. Examples of camptothecin derivatives include, but are not limited to, 9-nitro-20(S)-camptothecin, 9-amino-20(S)-camptothecin, 9-methyl-camptothecin, 9-chlorocamptothecin, 9-flouro-camptothecin, 7-ethyl camptothecin, 10-methylcamptothecin, 10-chloro-camptothecin, 10-bromo-camptothecin, 10-fluoro-camptothecin, 9-methoxy-camptothecin, 11-fluoro-camptothecin, 7-ethyl-10-hydroxy camptothecin, 10,11-methylenedioxy camptothecin, and 10,11-ethylenedioxy camptothecin, and 7-(4-methylpiperazinomethylene)-10,11-methylenedioxy camptothecin. Prodrugs of camptothecin include, but are not limited to, esterified camptothecin derivatives as described in U.S. Pat. No. 5,731,316, such as camptothecin 20-O-propionate, camptothecin 20-O-butyrate, camptothecin 20-O-valerate, camptothecin 20-O-heptanoate, camptothecin 20-O-nonanoate, camptothecin 20-O-crotonate, camptothecin 20-O-2′,3′-epoxy-butyrate, nitrocamptothecin 20-O-acetate, nitrocamptothecin 20-O-propionate, and nitrocamptothecin 20-O-butyrate. Particular examples of 20(S)-camptothecins include 9-nitrocamptothecin, 9-aminocamptothecin, 10,11-methylendioxy-20(S)camptothecin, topotecan, irinotecan,7-ethyl-10-hydroxy camptothecin, or another substituted camptothecin that is substituted at least one of the 7, 9, 10, 11, or 12 positions. These camptothecins may optionally be substituted.

Substitutions may be made to the camptothecin scaffold, while still retaining activity. In certain embodiments, the camptothecin scaffold is substituted at the 7, 9, 10, 11, and/or 12 positions. Such substitutions may serve to provide differential activities over the unsubstituted camptothecin compound. Examples of substituted camptothecins include 9-nitrocamptothecin, 9-aminocamptothecin, 10,11-methylendioxy20(S)-camptothecin, topotecan, irinotecan, 7-ethyl-10-hydroxy camptothecin, or another substituted camptothecin that is substituted at least one of the 7,9, 10, 11, or 12 positions.

Native, unsubstituted, camptothecin can be obtained by purification of the natural extract, or may be obtained from the Stehlin Foundation for Cancer Research (Houston, Tex.). Substituted camptothecins can be obtained using methods known in the literature, or can be obtained from commercial suppliers. For example, 9-nitrocamptothecin may be obtained from SuperGen, Inc. (San Ramon, Calif.), and 9-aminocamptothecin may be obtained from Idec Pharmaceuticals (San Diego, Calif.). Camptothecin and various analogs may also be obtained from standard fine chemical supply houses, such as Sigma Chemicals.

d. Conjugation

The invention contemplates the use of a single polymer or chelator and the use of mixtures of different polymers or chelators. Different polymers include, for example, similar polymers of different lengths, as well as substantially different polymers. The invention includes the use of a drug conjugated to a single polymer or chelator and a drug conjugated to multiple different polymers or chelators. Similarly, the invention includes the use of two or more drugs, each conjugated to the same type of polymer or chelator, as well as mixtures of two or more drugs, each conjugated to a different polymer or chelator. In certain embodiments, two or more different drug moieties may be conjugated to a single polymer or chelator moiety.

Polymers may be associated with the drug molecules in any manner known or available to those skilled in the art. For example, a polymer can be associated with a drug through a covalent bond, such as a peptide bond, for example, or through charge-charge interactions, vander wahl forces, and the like. Covalent bonds may be generated synthetically or by genetic fusion to produce a recombinant polymer-drug fusion protein. Exemplary methods of conjugating a polymer to drug are described, for example, in U.S. Pat. Nos. 5,977,163, 6,262,107 and 6,441,025, U.S. patent application Ser. Nos. 60/013,184, 09/530,601, 60,159,135, 09/686,627, 60/190,429, 09/810,345, 60,277,705, and 09/956,237; and PCT Publication Nos. WO 99/49901, WO 97/33552, WO 01/26693, and WO 01/70275, which are incorporated by reference herein.

Those of ordinary skill in the art will readily understand that a polymer and a drug may be conjugated or associated directly or via a secondary molecule such as a linker or a spacer (see e.g. WO 01/70275, the technology therein can be applied to any drug conjugate, particularly to polyamino acid conjugates). Preferred linkers include those that are relatively stable to hydrolysis in the circulation. Exemplary linkers include amino acids, hydroxyacidsdiols, aminothiols, hydroxythiols, aminoalcohols, beta alanines, glycol and combinations of these. In addition, the drug may require modification prior to conjugation, such as the introduction of a new functional group, the modification of a preexisting functional group or the attachment of a spacer molecule.

Chemical coupling may be achieved using commercially available homo- or hetero-bifunctional cross-linking compounds, according to methods known and available in the art, such as those described, for example, in Hermanson, Greg T., Bioconjugate Techniques, Academic Press, Inc., 1995, and Wong, Shan S., Chemistry of Protein Conjugation and Cross-linking, CRC Press, 1991, both of which are hereby incorporated by reference.

Additional examples of how carriers may be linked to drugs or linkers are described in Hoffman et al., Biol. Chem. 370:575-582, 1989; Wiesmuller et al., Vaccine, 7:29-33, 1989; Wiesmuller et al., Int. J. Peptide Protein Res., 40:255-260, 1992; Defourt et al., Proc. Natl. Acad. Sci. 89:3879-3883, 1992; Tohokuni et al., J. Am. Chem. Soc., 116:395-396, 1994; Reichel, Chem. Commun., 2087-2088, 1997; Kamitakahara, Angew. Chem. Int. Ed. 37:1524-1528, 1998; Dullenkopf et al., Chem. Eur. J., 5:2432-2438, 1999; all of which are hereby incorporated by reference.

In certain embodiments, a polymer is conjugated to a drug by chemical conjugation, as described in U.S. Pat. No. 5,977,163. In this method, polyglutamic acid conjugates are prepared as a sodium salt, dialyzed to remove low molecular weight contaminants and excess salts, and then lyophilized.

In another embodiment, a polymer carrier of the invention is conjugated to a drug by chemical conjugation, essentially as described in the published PCT application, WO 01/26693 A2. According to this method, a polyglutamic acid polymer is covalently bonded to a drug by a direct linkage between a carboxylic acid residue of the polyglutamic acid and a functional group of the drug, or by an indirect linkage via one or more bifunctional groups. A drug may be linked to a polymer or linker by any linking method available in the art and according to methods well known to those skilled in the art, including those found, for example, in March, J., Advanced Organic Chemistry, Wiley Interscience, 4th ed., 1992.

In one embodiment, a polyglutamate carrier is coupled to a drug according to a method comprising the following steps:

(a) providing a protonated form of a polyglutamic acid polymer and a drug for conjugation thereto;

(b) covalently linking said drug to said polyglutamic acid polymer in an inert organic solvent to form a polyglutamic acid-drug conjugate;

(c) precipitating said polyglutamic acid-drug conjugate from solution by addition of an excess volume of aqueous salt solution; and

(d) collecting said conjugate as a protonated solid.

The protonated form of the polyglutamic acid polymer in step (a) is obtained by acidifying a solution containing the salt of the polyglutamic acid to be used as a starting material, and converting the salt to its acid form. After separating the solid by centrifugation, the solid is washed with water. The polyglutamic acid is then dried, preferably by lyophilization and preferably to a constant weight comprising between about 2% to about 21% water, between about 7% to about 21% water, or between 7% and 21% water, prior to conjugation to a desired drug (step (b)).

Conjugates may be produced in whole, or in part, using recombinant DNA technology, as is widely known and available in the art. For example, a polymer or drug, or both, may be produced by recombinant means and thereafter associated or conjugated. Alternatively, a single polypeptide, for example, comprising both the polymer and the drug may be produced as a fusion protein. Methods of constructing recombinant expression vectors are known in the art, as are methods of expressing recombinant polypeptides in a variety of organisms, such as bacteria and yeast. Such methods are described, for example, in U.S. patent application Serial No. 60/277,705.

The amount of drug conjugated per polymer is variable. At the lower end, the drug-polymer conjugate may comprise from about 1%, about 2%, about 3%, about 4%, about 5%, about 6%, about 7%, about 8%, about 9%, or about 10%, about 11%, about 12%, about 13%, about 14%, about 15%, about 16%, about 17%, about 18%, about 19%, about 20%, about 21% about 22%, about 23%, about 24%, to about 25% (w/w) of drug relative to the mass of the conjugate. At the high end, the drug-polymer conjugate may comprise from about 26%, about 27%, about 28%, about 29%, about 30%, about 31% about 32%, about 33%, about 34%, about 35%, about 36%, about 37%, about 38%, about 39%, about 40%, to about 50% or more (w/w) of drug relative to the mass of the conjugate.

Similarly, the number of molecules of drug conjugated per molecule of polymer can vary. At the lower end, the drug-polymer conjugate may comprise from about 1, about 2, about 3, about 4, about 5, about 6, about 7, about 8, about 9, about 10, about 11, about 12, about 13, about 14, about 15, about 16, about 17, about 18, about 19, to about 20 or more molecules of the drug per molecule of polymer. At the higher end, the drug-polymer conjugate may comprise from about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60 about 61, about 62, about 63, about 64, about 65, about 66, about 67, about 68, about 69, about 70, about 71, about 72, about 73, about 74, to about 75 or more molecules or more of drug per molecule of water soluble polymer.

It should be recognized that a polymer may be associated with one or more discrete or overlapping sites on a drug molecule. Similarly, in certain embodiments, a drug molecule may be associated with one or more discrete sites on a polymer. Accordingly, in certain embodiments, compositions of the invention include polymers associated with drugs through different sites on the drug, as well as drugs associated with polymers through different sites on the carrier. Different linkers may be used to direct association through different sites, or a single linker may be used, depending on the particular functional groups present at each site. In certain embodiments, the invention includes a composition comprising a mixture of one or more polymers associated with one or more drugs through one or more different or overlapping sites on each drug or polymer.

In one embodiment, DTPA-paclitaxel is synthesized as described in U.S. Pat. No. 5,977,163, which is hereby incorporated by reference in its entirety, according to the following procedure. To a solution of paclitaxel (100 mg, 0.117 mmol) in dry DMF (2.2 ml) was added diethylenetriaminepentaacetic acid anhydride (DTPA A) (210 mg, 0.585 mmol) at 0° C. The reaction mixture was stirred at 4° C. overnight. The suspension was filtered (0.2 μm Millipore filter) to remove unreacted DTPA anhydride. The filtrate was poured into distilled water, stirred at 4° C. for 20 min, and the precipitate collected. The crude product was purified by preparative TLC over C₁₈ silica gel plates and developed in acetonitrile/water (1:1). Paclitaxel had an R_{f} value of 0.34. The band above the paclitaxel with an R_{f} value of 0.65 to 0.75 was removed by scraping and eluted with an acetonitrile/water (1:1) mixture, and the solvent was removed to give 15 mg of DTPA-paclitaxel as product (yield 10.4%): mp:>226° C. dec. The UV spectrum (sodium salt in water) showed maximal absorption at 228 nm, which is also characteristic for paclitaxel. Mass spectrum: (FAB) m/e 1229 (M+H)⁺, 1251 (M+Na), 1267 (M+K). In the ¹H NMR spectrum (DMSO-d₆) the resonance of NCH₂ CH₂N and CH₂COOH of DTPA appeared as a complex series of signals at δ2.71-2.96 ppm, and as a multiplet at δ3.42 ppm, respectively. The resonance of C7-H at 4.10 ppm in paclitaxel shifted to 5.51 ppm, suggesting esterification at the 7-position. The rest of the spectrum was consistent with the structure of paclitaxel.

The sodium salt of DTPA-paclitaxel was also obtained by adding a solution of DTPA-paclitaxel in ethanol into an equivalent amount of 0.05 M NaHCO₃, followed by lyophilizing to yield a water-soluble solid powder (solubility>20 mg equivalent paclitaxel/ml).

2. Combinations

The drug conjugates may be used in combination with any other treatment or drug. In certain embodiments, the drug conjugates are used in combination with non-drug treatment or therapies, such as surgery, radiation, or heat treatment, for example. In other embodiments, the drug conjugates are administered in combination with another drug. Coadministered drugs may themselves be drug conjugates, or they may be non-conjugated forms of a drug. Administration may be sequentially or simultaneously. The conjugates may be administered in combination with one or more other drugs or treatments, or any combination thereof. In certain embodiments, the conjugate and other treatment or drug used in combination have different mechanisms of action, and they may act additively, cooperatively or synergistically to combat a disease. In other embodiments, the conjugate and other treatment or drug used in combination have the same or similar mechanisms of action, and they may also act additively, cooperatively or synergistically to combat a disease.

Chemotherapy agents are often used in combination with surgery to remove a tumor or cancerous lesion. A patient may be treated with a chemotherapeutic agent before surgery, for example, to reduce the tumor size prior to surgery, or a patient may be treated during surgery of post-operatively, for example, to kill any cancer cells remaining following surgery. Thus, the invention includes a combination treatment comprising both surgery and the administration of a conjugate of the invention, wherein the conjugate is administered before, during, or following surgery. Surgical methods and techniques are known to one of skill in the art.

Chemotherapy drugs are frequently used in combination with radiotherapy to treat tumors, and the invention contemplates the use of the conjugates in combination with radiation treatment. Combined chemotherapy and radiotherapy has been shown to improve the response and survival rate of cancer patients, but there is still a need to establish more effective ways to deliver these agents.

Radiotherapy in combination with conjugate treatment may be used to treat a variety of cancers, including cancers of the skin, tongue, larynx, brain, breast, uterine, cervix, and leukemia and lymphoma, for example. Any type of radiation therapy used to treat cancer may be used in combination with drug conjugates, according to the invention. Such types include, for example, photon radiation, such as X-rays, and gamma rays. Radiotherapy performed in combination with drug conjugates may also include the use of radiosensitizers to make tumor cells more likely to be damaged or radioprotectors to protect normal cells from radiation damage.

Radiation may be delivered by a variety of means. For example, external beam radiotherapy is used to focus radiation on a cancer site. Another technique for delivering radiation to cancer cells is to place radioactive implants directly in a tumor or body cavity, i.e. internal radiotherapy, such as brachytherapy, interstitial irradiation, and intracavitary irradiation. Other approaches to radiation therapy include intraoperative irradiation, a form of external irradiation performed during surgery, and particle beam radiation therapy using fast-moving subatomic particles to treat localized cancers, such as high linear energy transfer radiation. Radiolabeled antibodies may be used to deliver radiation directly to a cancer site targeted by the antibody, i.e. radioimmunotherapy. Methods of treating patients using radiotherapy are well known to those of skill in the art. Dosages and schedules depend upon a variety of factors, such as the type of cancer, the type of radiation, and the method of delivery, and may be readily determined by one of ordinary skill in the art.

Recently, it was demonstrated that the radiosensitizing effect of a paclitaxel was enhanced when the drug was delivered as a polyglutamate conjugate at an optimal concentration and maintained in the tumor for a prolonged period. While combined radiation and paclitaxel produced additive or sub-additive interaction when radiation preceded paclitaxel injection, combined radiation and PG-TXL produced synergistic interaction in a mammary MCa-4 tumor model. Radiation appeared to significantly increase tumor uptake of PG-TXL, suggesting a potential role of radiation-modulated antitumor activity of polymeric drugs. Furthermore, studies have also shown that tumor irradiation enhanced the distribution of PG-TXL given 24 h later to ovarian Oca-1 carcinoma implanted i.m. in C3Hf/Kam mice. Combined radiation and PG-TXL also produced a significantly greater tumor growth delay than treatment with radiation and paclitaxel, when both drugs were given at the same equivalent paclitaxel dose of 60 mg/kg 24 h after tumor irradiation. Ibid. Further studies demonstrated that the radiosensitizing effect of paclitaxel was enhanced when it was delivered systemically as a polymer-drug conjugate to Oca-1 tumors. Li, C., et al., Int. J. Radiat. Oncol. Biol. Phys. 48(4):1119-26 (2000). The radiosensitizing effect of PG-TXL was dependent on the interval between PG-TXL administration and radiation delivery, with greater enhancement observed when the interval was decreased. Ibid. These studies support a treatment strategy combining radiation and polymeric chemotherapy that may have important clinical implications in terms of scheduling and optimization of the therapeutic ratio. Ibid. These results may be extrapolated to other drug conjugates, particularly those demonstrating increased tumor uptake compared to the free drug.

Although not a routine cancer therapy, the use of heat therapy in combination with chemotherapy is increasingly being examined, since there is evidence that heat therapy may cause cancer cells to become more sensitive to conventional treatments. Thus, the invention contemplates using the conjugates in combination with heat therapy or hypothermia. The three major types of heat therapy contemplated are local, regional, and whole-body. Local hypothermia refers to heat being applied to very small areas, such as a tumor. Typically, the area is heated externally using high-frequency waves aimed at a tumor from a device outside the body. Internal heating may be accomplished using a sterile probe, such as a heated wire, implanted microwave antennae, or radiofrequency electrode, for example. Regional hypothermia involved treating an organ or limb. Methods of regional hypothermia include placing magnets or other high energy devices over the region to be heated and perfusion, wherein patient's blood is removed, heated, and perfused into the region to be heated. Whole body heating is typically used to treat metastatic cancer that has spread throughout the body. It can be accomplished using warm blankets, hot wax, inductive coils, or thermal chambers, for example. Hypothermia treatment protocols are known by and available to one of ordinary skill in the art.

The invention includes using the conjugates in combination with other drugs, such as chemotherapeutic or anti-proliferative drugs. Typically, anticancer drugs destroy cancer cells by stopping them from growing or multiplying at one or more points in their growth cycle. Chemotherapy may consist of one or several cytotoxic drugs, depending on the type of cancer being treated. Goals of chemotherapy include shrinking primary tumors, slowing tumor growth, and killing cancer cells that may have spread (metastasized) to other parts of the body from the original tumor. However, chemotherapy kills both cancer and healthy cells, and it is necessary to try to minimize damage to normal cells and enhance the cytotoxic effect to cancer cells.

One means of potentially optimizing chemotherapy is by using a combination of chemotherapy drugs. Combination therapy using drugs with similar or the same mechanisms of action may allow lower doses of one or more drug to be used, potentially reducing toxic side-effects associated with a drug formulation. Combination therapy using drugs with different mechanisms of action may be more effective at killing cells, since they attack or weaken the cell at multiple levels or sites. Use of the conjugates according to the invention in combination therapy offers additional advantages. For example, since the conjugates are more readily adsorbed and more stable than free drug, lower doses may be used to achieve the same drug concentration or therapeutic effect. Furthermore, since the conjugates are typically more water-soluble than free drugs, the need to use toxic delivery vehicles, such as Cremophor, is reduced. Therefore, higher doses of the conjugate may be delivered as compared to the free drug formulation containing a toxic vehicle or component.

Certain antineoplastic agents are recognized to act synergistically. The metabolic basis of synergy, for example, between 5 fluorouracil and methotrexate is understood, although the mechanism of synergy between other drugs, however, is not so clear. Certain drugs can be assigned as having chiefly S-phase or M-phase activity, and a possible explanation emerges regarding their synergistic action. Agents acting on targets that are sequential in the cell cycle would be expected to act in synergy: an agent that acts in S-phase might be expected to synergize with M-phase agents. Using this rationale, many chemotherapeutic protocols can be shown to be combinations of S-phase and M-phase agents, for example. Discussed in U.S. Pat. No. 6,150,398, which is hereby incorporated by reference in its entirety.

Examples of approved oncology drugs that may be used in combination with a drug conjugated to a polymer or chelator include, but are not limited to, antimetabolites, nucleoside analogs, signal transduction inhibitors, azacytidine, adriamycin, alkeran, allopurinol, altretamine, amifostine, anastrozole, araC, arsenic trioxide, azathioprine, bexarotene, biCNU, bleomycin, busulfan intravenous, busulfan oral, camptothecins, capecitabine (Xeloda), carboplatin, carmustine with Polifeprosan 20 Implant, CCNU, celecoxib, chlorambucil, cisplatin, cisplatin-epinephrine gel, cladribine, cyclosporin A, cytarabine liposomal, cytosine arabinoside, daunorubicin liposomal, cytoxan, daunorubicin, dexrazoxane, dodetaxel, doxorubicin, doxorubicin liposomal, DTIC, Elliott's B Solution, epirubicin, estramustine, etoposide phosphate, etoposide and VP-16, exemestane, FK506, fludarabine, fluorouracil, 5-FU, gemcitabine (Gemzar), gemtuzumab-ozogamicin, goserelin acetate, hydrea, hydroxyurea, herceptin, idarubicin, ifosfamide, imatinib mesylate, interferon, irinotecan (Camptostar, CPT-111), irressa, letrozole, leucovorin, leustatin, leuprolide, levamisole, liposomal daunorubicin, litretinoin, megastrol, melphalan, L-PAM, mesna, methotrexate, methoxsalen, mithramycin, mitomycin, mitoxantrone, vinorelbine, nitrogen mustard, oxaloplatin, paclitaxel, pamidronate, Pegademase, pentostatin, porfimer sodium, rituxan, streptozocin, STI-571, talc, tamoxifen, taxotere, temozolamide, teniposide, VM-26, topotecan (Hycamtin), toremifene, tretinoin, ATRA, valrubicin, velban, vinblastine, vincristine, VP16, and vinorelbine.

Uses of these in combination with drug-polymer conjugates include, but are not limited to, the use of single or multiple drug-polymer conjugates with single and/or multiple non-conjugated oncology drugs. Examples include, but are not limited to, the use of PG-TXL+carboplatin +gemcitibine, PG-TXL+carboplatin, PG-TXL+gemcitibine, etc. Preferred treatments include the use of 175, 210, 225, 235 or 250 mg/m2 paclitaxel equivalent PG-TXL (preferred dosing of infusion over ten minutes once every three weeks) with carboplatin (AUC=5 or 6; preferred dosing of 30 minute IV infusion on the same day and before or after PG-TXL dosing) and/or cisplatin (75 or 80 mg/m2; preferred dosing of 3 hour IV infusion on the same day and before or after PG-TXL dosing) and/or gemcitabine (1250 mg/m2) every three weeks and/or vinorelbine (30 mg/m2) weekly. Other preferred dosages include once weekly dosing of 4,25 or 50 mg/m2, and any combinations thereof. In the once weekly dosing, 50-100 mg/m2 is preferred. Preferred treatment cycles are until disease progression, thee cycles after remission or until death. Any of these combination for use with or without radiation therapy is also contemplated.

It is also contemplated that the use of drug-polymer conjugates reduces the toxicities of chemotherapy, increasing the amounts of conjugated drug, as well as unconjugated drug, that can be administered in a combination therapy. For example, some combinations of gemcitabine are limited to the use of 10 and 20 micromolar amounts. Combination with polymer-conjugates (e.g., PG-TXL) with gemcitabine increases the amount of gemcitabine that can be safely administered (i.e., more than 20 micromolar) as well as the conjugated paclitaxel.

Examples of drugs used in combination with conjugates and other chemotherapeutic agents to combat undesirable side effects of cancer or chemotherapy include zoledronic acid (Zometa) for prevention of bone metastasis and treatment of high calcium levels, Peg-Filgrastim for treatment of low white blood count, SDZ PSC 833 to inhibit multidrug resistance, and NESP for treatment of anemia.

In one embodiment of the invention, drug conjugates used in combination with other drugs are conjugates of a taxoid to a polymer, such as PG-TXL, for example. Paclitaxel and docetaxel have each been used in combination with other drugs. Paclitaxel in combination with other anticancer drugs may be used to treat many different types of cancer, including lymphoma and cancers of the head and neck, breast, esophagus, stomach, bladder, prostate, endometrium (uterus), and cervix, for example. Clinical trials are underway to test the effectiveness of docetaxel, in combination with other cancer drugs, for treatment of a variety of cancers, including cancers of the head and neck, prostate, breast, lung, and endometrium (uterus), for example. The effectiveness of paclitaxel-based combination therapy has been limited in some instances by adverse side effects and limited adsorption. The use of a conjugated form of paclitaxel offers the advantages of lower toxicity and increased solubility and absorption.

Specific combinations used according to the invention include, for example, the use of a conjugate in place of, or in addition to, the corresponding free drug. In certain embodiments of the invention, conjugates of a taxoid or taxane, such as paclitaxel or docetaxel, with a polymer or chelator are used in place of a free taxoid or taxane in combination therapy. Paclitaxel has been combined therapeutically within two drug combinations with a platinum, such as cisplatin, oxaliplatin, or carboplatin, raltitrexed (TOMUDEX®), high-dose cyclophosphamide, vinorelbine tartrate, R115777, flavopiridol, ER-51785, ZD1839 (IRESSA), and gemcitibine, for example. Paclitaxel has also been used in three or more drug combinations with a platinum and anthracycline, gemcitabine and doxorubicin, OSI-744, and carboplatin, gemcitibine and carboplatin, topotecan and carboplatin, the tyrosine kinase inhibitor PKI166 and ST1571 (imatinib mesylate, GLEEVEC™), epirubicin and cisplatin, cisplatin and fluorouracil, mesna and cisplatin, and epirubicin and carboplatin, for example. In addition, paclitaxel has been used in combination with bleomycin, etoposide, and cisplatin and in a combination referred to as ICE-T, which includes ifosfamide, carboplatin, etoposide, paclitaxel, and mesna. Dodetaxel has been used in combination with cyclosporine, OSI-774, CPT-111, trastuzumab, doxorubicin, capecetabine, cisplatin and 5-fluorouracil, and gemcitabine, for example.

3. Formulations

A composition according to the present invention may further comprise components or substances that are useful in formulating the composition. Carriers for therapeutic use are well known, and are described, for example, in Remingtons Pharmaceutical Sciences, Mack Publishing Co. (A. R. Gennaro ed. 1985). In general, the type of carrier is selected based on the mode of administration. Pharmaceutical compositions may be formulated for any appropriate manner of administration, including, for example, topical, oral, nasal, intrathecal, rectal, vaginal, sublingual or parenteral administration, including subcutaneous, intravenous, intramuscular, intrasternal, intracavernous, intrameatal or intraurethral injection or infusion.

Accordingly, the substances suitable for the present invention include, but are not limited to, physiologically acceptable excipients, diluents, and additive agents such as an acidic salt, a basic salt, a neutral salt, a carbohydrate, a starch, a polyelectrolyte, biocompatible hydrophilic materials, swellable materials, a gelatin, an amine, a surfactant, an inorganic acid or base, an organic acid or base, an amino acid, a monomer, an oligomer, a polymer or a mixture thereof. Physiologically acceptable excipients, such as vehicles, adjuvants, carriers or diluents, are readily available to the public. Moreover, physiologically acceptable auxiliary substances, such as pH adjusting and buffering agents, tonicity adjusting agents, stabilizers, wetting agents and the like, are readily available to the public.

In certain embodiments, the substance may include, but is not limited to, sodium chloride, sodium phosphate, bile salts, ammonium sulfate, ammonium chloride, sodium carbonate or potassium carbonate, polyethylene glycol, polyoxoethylene alkyl ethers, trehalose, mannitol, sorbitol, dextrose, dextrin, sucrose, lactose, saccharides, polysaccharides, oligosaccharides, saccharin, carboxymethyl cellulose, hydroxypropyl cellulose, hydroxypropyl methyl cellulose, methyl cellulose or sodium starch glycolate, citric acid, lactic acid, glycolic acid, acetic acid, ascorbic acid, tartaric acid, malic acid, maleic acid, benzoic acid, arginine, glycine, threonine, choline, ethanolamine, protamine, sodium alginate, heparin, docusate sodium, glycerin, glycofurol, propylene glycol, polysorbate, povidone, or albumin.

Another component optionally for use in the composition of the present invention is a metabolizable, non-toxic oil, preferably one of 6 to 30 carbon atoms, including, but not limited to, alkanes, alkenes, alkynes, and their corresponding acids and alcohols, the ethers and esters thereof, and mixtures thereof. The oil may be any vegetable oil, fish oil, animal oil, or synthetically prepared oil which can be metabolized by the body of the subject to which the adjuvant will be administered and which is not toxic to the subject.

The optional oil component of this invention may be any long chain alkane, alkene, or alkyne, or an acid or alcohol derivative thereof, either as the free acid, its salt or an ester such as a mono-, di- or triester, such as the triglycerides and esters of 1,2-propanediol or similar poly-hydroxy alcohols. Alcohols may be acylated employing a mono- or poly-functional acid, for example, acetic acid, propanoic acid, citric acid or the like. Ethers derived from long chain alcohols, which are oils and meet the other criteria set forth herein may also be used.

In certain embodiments, the aqueous portion of the immunogenic compositions is buffered saline. When these compositions are intended for parenteral administration, it is preferable to make up these solutions so that the tonicity, i.e., osmolality, is essentially the same as normal physiological fluids in order to prevent post-administration swelling or rapid absorption of the composition because of differential ion concentrations between the composition and physiological fluids. It is also preferable to buffer the saline in order to maintain a pH compatible with normal physiological conditions. Also, in certain instances, it may be necessary to maintain the pH at a particular level in order to insure the stability of certain composition components such as the glycopeptides. The pH of the aqueous component will generally be between 6.0-8.0 though it may be advantageous to adjust the pH of the system to 6.8, where this pH does not significantly reduce the stability of other composition components and is not otherwise physiologically unsuitable.

In certain embodiments, a composition of the present invention may comprise a surfactant. The term “surfactant” refers to non-toxic surface active agents capable of stabilizing an emulsion. There are a substantial number of emulsifying and suspending agents generally used in the pharmaceutical sciences. These include naturally derived materials such as gums, vegetable protein, alginates, cellulose derivatives, phospholipids (whether natural or synthetic), and the like. Certain polymers having a hydrophilic substituent on the polymer backbone have surfactant activity, for example, povidone, polyvinyl alcohol, and glycol ether-based compounds. Compounds derived from long chain fatty acids are a third substantial group of emulsifying and suspending agents usable in this invention. Though any of the foregoing surfactants can be used so long as they are non-toxic, glycol ether-based surfactants and non-ionic surfactants are preferred. Non-ionic surfactants include, for example, polyethylene glycols (especially PEG 200, 300, 400, 600 and 900), SPAN®, ARLACEL®, TWEEN®, MYRJ®, BRIJ® (all available from ICI America Inc., Wilmington, Del.), polyoxyethylene, polyol fatty acid esters, polyoxyethylene ether, polyoxypropylene fatty ethers, bee's wax derivatives containing polyoxyethylene, polyoxyethylene lanolin derivatives, polyoxyethylene fatty glycerides, glycerol fatty acid esters or other polyoxyethylene acid alcohol or ether derivatives of long-chain fatty acids of 12-21 carbon atoms. The presently preferred surfactant is TWEEN® 80 (otherwise known as polysorbate 80 or polyoxyethylene 20 sorbitan monooleate), although it should be understood that any of the above-mentioned surfactants would be suitable after lack of toxicity is demonstrated.

A surfactant may be added to the processing media and/or to a solution of the polymeric or chelator drug composition. The residue of such a surfactant will typically remain in the polymeric composition upon formation of an encapsulated agent. The surfactant can be cationic, anionic or nonionic. Examples of useful surfactants include but are not limited to carboxymethyl cellulose, gelatin, poly(vinyl pyrrolidone), poly(ethylene glycol), Tween 80, Tween 20, polyvinyl alcohol or mixtures thereof. The surfactant, preferably, should not hinder the biodegradation of the polymeric composition and release of the conjugated drug.

Compositions may be prepared as injectables, as liquid solutions or emulsions. The compositions may be mixed with physiologically acceptable excipients which are compatible with the compositions, including polymer and chelator conjugates. Examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity-enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer, and Tris buffer, while examples of preservatives include thimerosal, o-cresol, formalin, and benzyl alcohol. Standard formulations can either be liquids or solids that can be taken up in a suitable liquid as a suspension or solution for administration to an animal. Thus, in a non-liquid formulation, the excipient can comprise dextrose, human serum albumin, preservatives. etc., to which sterile water or saline can be added prior to administration.

For oral preparations, the conjugates can be used alone or in combination with appropriate additives to make tablets, powders, granules or capsules, for example, with conventional additives, such as lactose, mannitol, corn starch or potato starch; with binders, such as crystalline cellulose, cellulose derivatives, acacial, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

In certain embodiments, the conjugates and drugs may be formulated into preparations for injections by dissolving, suspending or emulsifying them in an aqueous or nonaqueous solvent, such as vegetable or other similar oils, synthetic aliphatic acid glycerides, esters or higher aliphatic acids or propylene glycol; and if desired, with conventional additives such as solubilizers, isotonic agents, suspending agents, emulsifying agents, stabilizers and preservatives.

In certain embodiments, a conjugate or drug can be utilized in aerosol formulation to be administered via inhalation. The compounds of the present invention can be formulated into pressurized acceptable propellants such as dichlorodifluoromethane, propane, nitrogen and the like.

In certain embodiments, a conjugate or drug may be formulated into an implant. Implants for sustained release formulations are well known in the art. Implants are formulated as microspheres, slabs, etc. with biodegradable or non-biodegradable polymers. For example, polymers of lactic acid and/or glycolic acid form an erodible polymer that is well-tolerated by the host. The implant is placed in proximity to the site of response, where applicable, so that the local concentration of active agent is increased relative to the rest of the body.

The compositions described herein may be formulated for sustained release (i.e., a formulation such as a capsule or sponge that effects a slow release of compound following administration). Such compositions may generally be prepared using well-known technology and administered by, for example, oral, rectal or subcutaneous implantation, or by implantation at the desired target site. Sustained-release formulations may contain an agent dispersed in a carrier matrix and/or contained within a reservoir surrounded by a rate controlling membrane. Carriers for use within such formulations are biocompatible, and may also be biodegradable; preferably the formulation provides a relatively constant level of active component release. The amount of active compound contained within a sustained release formulation depends upon the site of implantation, the rate and expected duration of release and the nature of the condition to be treated or prevented.

In certain embodiments, a controlled release formulation comprises a biodegradable polymer microspheres or microparticles wherein a conjugate is suspended in a polymer matrix, the polymer matrix being formed from at least two highly water soluble biodegradable polymers, and the microspheres being coated with a (d, 1 lactide-glycolide) copolymer.

In one embodiment, the polymers are selected from the group consisting of starch, crosslinked starch, ficoll, polysucrose, polyvinyl alcohol, gelatine, hydroxymethyl cellulose, hydroxyethyl cellulose, hydroxypropyl cellulose, hydroxypropyl-ethyl cellulose, hydroxypropyl-methyl cellulose, sodium carboxymethyl cellulose, cellulose acetate, sodium alginate, polymaleic anhydride esters, polyortho esters, polyethyleneimine, polyethylene glycol, methoxy-polyethylene glycol, ethoxypolyethylene glycol, polyethylene oxide, poly(1,3 bis(p-carboxyphenoxy) propane-co-sebacic anhydride, N,N-diethylaminoacetate, block copolymers of polyoxyethylene and polyoxypropylene. An example of a suitable polyortho ester is 3,9-bis(methylene)-2,4,8,10,-tetra oxaspiro[5,5]undecane/1,6 hexanediol poly (ortho ester). It is preferred that the weight ratio of the two polymers is in the range of from 20:80 to 80:20.

In another embodiment, the polymer matrix is selected from starch and ficoll, starch and polysucrose, starch and polyvinyl alcohol, starch and gelatine, hydroxyethyl cellulose and hydroxypropyl cellulose, gelatine and hydroxyethyl cellulose, gelatine and polyvinyl alcohol, polysucrose and polyvinyl alcohol, and sodium carboxymethyl cellulose and sodium alginate. When the polymer matrix comprises starch and ficoll, the preferred weight ratio of starch to ficoll is preferably from 85:15 to 60:40, and more preferably from 75:25 to 65:35.

Partially synthetic cellulose esters, polyvinylpyrrolidone and poly-6-aminohexanoic acid as well polyvinylalcohol, alkali and ammonium alginate, methylcellulose, ethylcellulose, hydroxyethylcellulose, ethylhydroxyethylcellulose, and sodium-carboxymethylcellulose have particularly proven their value as water-soluble polymers, which are to be used pursuant to the invention.

The selection of the particular (d, l lactide-glycolide) copolymer will depend in a large part on how long a period the microsphere is intended to release the active ingredient. For example, a (d, 1 lactide-glycolide) copolymer made from about 80% lactic acid and 20% glycolic acid is very stable and would provide a microsphere suitable for release of active ingredient over a period of weeks. A (d, 1 lactide-glycolide) copolymer made from 50% lactic acid and 50% glycolic acid is stable and would provide an microsphere suitable for release of active ingredient over a period of days. A (d, 1 lactide-glycolide) copolymer made from 20% lactic acid and 80% glycolic acid disintegrates relatively easily and would provide an microsphere suitable for release of active ingredient over a period of 1-2 days. The coating makes the microspheres more resistant to enzymatic degradation.

In the compositions of this invention, the component(s) of the polymeric composition are preferably biocompatible, which term is known in the art to include that the components are substantially non-toxic, non carcinogenic, and should not substantially induce inflammation in body tissues upon administration.

The biodegradable polymer is used in an amount ranging from 1 to 100, typically, from 5 to 30 times the weight of the core particle. The coating of the core particle is made of a water-soluble substance that is insoluble in the organic solvent. Exemplary hydrophobic biodegradable polymers which may be used in the present invention include poly(lactide-co-glycolide)(PLGA), polyglycolide(PGA), polylactide(PLA), copolyoxalates, polycaprolactone, poly(lactide-co-caprolactone), polyesteramides, polyorthoesters, poly(β-hydroxybutyric acid), and polyanhydride; while PLGA and PLA are preferred.

Any of the organic solvents well-known in the art may be used to dissolve the biodegradable polymer, and these include carbon tetrachloride, methylene chloride, acetone, chloroform, ethyl acetate and acetonitrile. General techniques for the preparation of conjugate encapsulated structures are known to those of skill in the art. See, e.g., U.S. Pat. No. 5,407,609 to Tice et al. and European Patent No. A1 0,058, 481 to Hutchinson.

In certain embodiments, a drug conjugate of the present invention can be formed into a core particle that may be coated by a biodegradable polymer, as described in U.S. Pat. No. 5,753,234, which is incorporated herein by reference in its entirety. The core particle is prepared by dissolving or dispersing the drug conjugate in a solution obtained by dissolving a water-soluble substance in a suitable aqueous solvent, e.g., water or a buffer, and drying the mixture by a spray drying or a freeze drying method.

The water-soluble substance used for the preparation of the core particle does not bring about an undesirable interaction with the drug conjugate and is practically insoluble in the organic solvent used in the coating step. Any water-soluble substance is contemplated so long that it does not bring into the composition any undesirable effects, such as local toxicity, for example. Exemplary water-soluble substances include water-soluble saccharides such as glucose, xylose, galactose, fructose, lactose, maltose, saccharose, alginate, dextran, hyaluronic acid, chondroitin sulfate and water-soluble cellulose derivatives, e.g., hydroxypropylmethyl cellulose, hydroxypropyl cellulose (HPC), carboxymethyl cellulose (CMC) and sodium carboxymethyl cellulose (CMC-Na); amino acids such as glycine, alanine, glutamic acid, arginine, lysine and a salt thereof; and a mixture thereof; while HPC, CMC, CMC-Na, gelatin, and a mixture thereof are preferred.

The water-soluble substance may be used in an amount ranging from 1 to 50, preferably, from 5 to 15 times the weight of total antigen.

The core particle so prepared has a particle size ranging from 0.1 to 200 μm, preferably, from 0.5 to 30 μm. In order to prepare the final microparticle, the core particle is dispersed in an organic solvent, wherein a hydrophobic biodegradable polymer is dissolved, by using a suitable apparatus, e.g., a magnetic stirrer, homogenizer, microfluidizer and sonicator.

Specifically, a microparticle of the present invention may be prepared from the core particle dispersed system in accordance with any one of the following conventional methods.

1) Solvent Evaporation Method

This method is well known for the preparation of a microparticle, but the present invention differs from the prior arts in that the core particle dispersed system, wherein the contact of the antigen with the organic solvent is prevented, is employed in place of an aqueous solution wherein the antigen is dissolved or dispersed.

Specifically, the microparticle may be prepared by dispersing the core particle dispersed system in an aqueous solution comprising a surfactant to obtain an O/W emulsion and then removing the organic solvent from the core particle dispersed system, or by dispersing the core particle dispersed system in a solvent, which is immiscible with the core particle dispersed system and is a nonsolvent for the biodegradable polymer, to prepare an O/O emulsion and removing the organic solvent from the core particle dispersed system. When acetonitrile is used as the organic solvent of the core particle dispersed system, a mineral oil can be used as the solvent which is immiscible with the core particle dispersed system and is a nonsolvent for the biodegradable polymer

2) Solvent Extraction Method

This method is also well-known in the art for the preparation of a microparticle, but the present invention differs from the prior arts in that the core particle dispersed system is employed. Specifically, the microparticle may be prepared by extracting the organic solvent of the core particle dispersed system by using a solvent, which is immiscible with the core particle dispersed system and is a nonsolvent for the biodegradable polymer, such as mineral oil or paraffin oil.

3) Rapid Freezing and Solvent Extraction Method

The present invention is different from the prior arts in that the core particle dispersed system is employed. Specifically, the core particle dispersed system is sprayed into a low-temperature liquid gas phase using an ultrasonic apparatus to form a frozen particle. This particle is collected on the surface of frozen ethanol. As the frozen ethanol is melted, the frozen particle thaws and the organic solvent in the particle is extracted into the ethanol phase with concomitant formation of a microparticle coated with the biodegradable polymer.

4) Spray Drying Method

This method is most preferable for use in the present invention and, specifically, the microparticle is prepared by spraying the core particle dispersed system by employing a spray-dryer. This method is advantageous due to its high productivity and rapidity. Further, it is also advantageous in that removal of water is unnecessary because water is not used in the process; no surfactant is required; and the washing and drying processes can be omitted.

The particle size of the microparticle thus prepared ranges from 0.5 to 300 μm, preferably, from 1 to 180 μm. Those microparticles having a particle size smaller than 180 μm may be dispersed in an injection medium to prepare an injection formulation for subcutaneous, intramuscular, and intraperitoneal injections. Those particles having a particle size larger than 180 μm may be used for preparing a formulation for oral administration.

The invention further provides a single-shot formulation that is prepared by dispersing the microparticles in a suitable injection medium. The formulation may comprise a single drug conjugate, or it may comprise one or more additional drugs or conjugates. A formulation comprising two or more conjugates or drugs may be prepared by employing core particles comprising a mixture of two or more kinds of conjugates or drugs, or by employing a mixture of two or more kinds of core particles each comprising a different conjugate or drug.

Within a pharmaceutical composition, a conjugate may itself be linked to any of a variety of compounds. For example, a polymer-drug conjugate may be linked to a targeting moiety (e.g., a monoclonal or polyclonal antibody, a protein or a liposome) that facilitates the delivery of the agent to a target site, such as a particular cell or tissue type. As used herein, a “targeting moiety” may be any substance (such as a compound or cell) that, when linked to a conjugate, enhances the transport of the conjugate to a target cell or tissue, thereby increasing the local concentration of the conjugate. Targeting moieties include antibodies or fragments thereof, receptors, ligands and other molecules that bind to cells of, or in the vicinity of, the target tissue. An antibody targeting agent may be an intact (whole) molecule, a fragment thereof, or a functional equivalent thereof. Examples of antibody fragments are F(ab′)₂, -Fab′, Fab and F[v] fragments, which may be produced by conventional methods or by genetic or protein engineering. Linkage is generally covalent and may be achieved by, for example, direct condensation or other reactions, or by way of bi- or multi-functional linkers. Targeting moieties may be selected based on the cell(s) or tissue(s) toward which the conjugate is expected to exert a therapeutic benefit.

B. Methods

1. Treatment of Cancer and Other Diseases

One or more conjugates of the invention, such as PG-TXL, for example, may be used in a combination with another treatment or drug to treat a patient. As used herein, a “patient” refers to any mammal, including a human, and may be afflicted with any disease or condition that may be effectively treated with a drug, including a drug conjugate. Treatment with the conjugate may be a primary treatment for a disease or a disorder, or it may be adjuvant therapy for a disease or disorder. Furthermore, the treatment may be of an existing disease or may be prophylactic.

a. Diseases

Compositions of the invention may be used to treat a variety of diseases and disorders, including cancers and tumors. Conditions that may be treated with a combination therapy including a conjugate according to the invention include disorders associated with cell proliferation, including Duchenne muscular dystrophy, cancer, graft-versus-host disease (GVHD), autoimmune diseases, allergy or other conditions in which immunosuppression may be involved, metabolic diseases, abnormal cell growth or proliferation and cell cycle abnormalities. Preferable indications that may be treated using the combination therapies of the present invention include those involving undesirable or uncontrolled cell proliferation. Such indications include restenosis, benign tumors, a various types of cancers such as primary tumors and tumor metastasis, abnormal stimulation of endothelial cells (atherosclerosis), insults to body tissue due to surgery, abnormal wound healing, abnormal angiogenesis, diseases that produce fibrosis of tissue, repetitive motion disorders, disorders of tissues that are not highly vascularized, and proliferative responses associated with organ transplants.

Specific types of restenotic lesions that can be treated using the present invention include coronary, carotid, and cerebral lesions. Specific types of benign tumors that can be treated using the present invention include hemangiomas, acoustic neuromas, neurofibroma, trachomas and pyogenic granulomas. Specific types of cancers that can be treated using this invention include acute myelogenous leukemia, bladder, breast, cervical, cholangiocarcinoma, chronic myelogenous leukemia, colon, colorectal, esophagial, fallopian tube, gastric sarcoma, glioma, glioblastoma, head and neck, Kaposi's sarcoma, leukemia, lung (e.g. non-small cell lung cancer), lymphoma, melanoma, multiple myeloma, osteosarcoma, ovarian (e.g. epithelian ovarian), peritoneal carcinoma, pancreatic, prostrate, solid tumors, stomach, or tumors at localized sites including inoperable tumors or in tumors where localized treatment of tumors would be beneficial, and solid tumors. Treatment of cell proliferation due to insults to body tissue during surgery may be possible for a variety of surgical procedures, including joint surgery, bowel surgery, and cheloid scarring.

Proliferative responses associated with organ transplantation that may be treated using this invention include those proliferative responses contributing to potential organ rejections or associated complications. Specifically, these proliferative responses may occur during transplantation of the heart, lung, liver, kidney, and other body organs or organ systems.

In certain embodiments of the invention, a combination comprising a drug conjugate is used to treat a disease or condition wherein effective treatment is limited by toxic side effects associated with a chemotherapeutic agent. In addition, the invention may be used to treat a condition or disease wherein the efficacy of a drug is limited by one or more pharmaceutical characteristics, such as water insolubility or poor cellular or tissue uptake, for example.

In certain embodiments, particularly when the drug conjugate comprises a taxoid or taxane, such as paclitaxel, for example, combinations of the invention may be used to treat a tumor responsive to taxoid or taxane treatment. Examples of tumors that may be responsive to treatment with a taxoid or taxane, such as paclitaxel, include ovarian cancer, breast cancer, lymphoma, leukemia, cancers of the head and neck, esophagus, stomach, bladder, prostate, endometrium, and cervix.

Camptothecins, and therefore, camptothecin conjugates may also be used in combination therapy, according to the invention to treat a variety of proliferation-associated diseases. Camptothecins have previously been used to treat a number of tumors, including, for example, ovarian, colon, colorectal, lung, pancreatic, and gastric cancers, as well as leukemia. Accordingly, the invention contemplates the use of camptothecin conjugates to treat these and other diseases.

b. Administration

The compositions of the present invention can be administered to a subject using a variety of methods known in the art. In one embodiment, the conjugate can be delivered parenterally, by injection, such as intramuscular, intraperitoneal, intravenous or subcutaneous injection, or by inhalation. In other embodiments, the conjugate can be delivered rectally, vaginally, nasally, orally, opthamalically, topically, transdermally or intradermally. When the mode of administration is by injection of an encapsulated conjugate, the encapsulated conjugate may stay at the injection site for up to two weeks or longer, thus providing a depot of drug that will give sustained release or pulsatile release in vivo. Such a delivery system may allow single-shot formulations to be produced for drugs that would otherwise require multiple injections to elicit a beneficial response.

c. Dosage and Schedule

Many oncologists have experience working with single taxane skeleton molecules, such as paclitaxel and docetaxel. While the chemical structures of paclitaxel and docetaxel provide a single taxane skeleton per molecule, many taxane conjugates, such as PG-TXL have multiple taxane skeletons per molecule. In order to capitalize on the prior experience of treating physicians and to provide at least one meaningful comparison between multiple taxane skeleton molecule therapies and older single taxane skeleton molecule therapies, the dosing of taxane conjugates can be expressed as “paclitaxel equivalents”. For example, for dosing purposes, each taxane skeleton in a taxane conjugate molecule is calculated as one paclitaxel equivalent.

Pharmaceutical compositions may be administered in a manner appropriate to the disease to be treated (or prevented). An appropriate dosage and a suitable duration and frequency of administration will be determined by such factors as the condition of the patient, the type and severity of the patient's disease, the particular form of the active ingredient and the method of administration. In general, an appropriate dosage and treatment regimen provides the agent(s) in an amount sufficient to provide therapeutic and/or prophylactic benefit (e.g., an improved clinical outcome, such as more frequent complete or partial remissions, or longer disease-free and/or overall survival). For prophylactic use, a dose should be sufficient to prevent, delay the onset of or diminish the severity of the disease being treated or prevented.

When used in combination therapy in place of a free drug, a drug conjugate according to the invention may be used at any equivalent dose used for the free drug. In addition, the invention includes using both lower and higher equivalent doses than those used for a free drug, for any given situation or tumor type. Since the drug conjugates are typically more stable, more soluble, or more readily taken up by a tumor, similar therapeutic effects may be accomplished using a lower equivalent dose, as compared to free drug. Similarly, since the drug conjugates are typically less toxic than pharmaceutical formulations of free drug, the invention includes using a higher equivalent dose, as compared to the free drug.

According to the invention, the drug conjugates are used in combination with another therapy or drug. Where the conjugate is used in combination with one or more other drugs, another drug may be used at higher or lower doses than typically used when it is combined with the free drug of the coadministered conjugate. For example, where the free drug of the coadministered conjugate and the other drug share a toxicity or side effect, and the conjugated form of the drug exhibits reduced toxicity, it may be possible to use a higher dose of the other drug in combination with the conjugate, as opposed to the free drug.

The exact amount of such compositions required will vary from subject to subject, depending on the species, age, weight and general condition of the subject, the severity of the disease, infection or condition that is being treated or prevented, the particular compound used, its mode of administration, and the like. Thus, it is not possible to specify an exact amount. However, an appropriate amount may be determined by one of ordinary skill in the art using only routine experimentation given the teachings herein. A single administration may be sufficient, depending upon the disease, condition, or infection being treated or prevented; however, it is also contemplated that multiple administrations may be administered. Administrations after the initial administration may be of higher or lower dosage than the initial dosage. Similarly, the amount of one or more of the conjugates or drugs used in combination therapy may be altered for different doses throughout a course of treatment.

Optimal dosages may generally be determined using experimental models and/or clinical trials. The use of the minimum dosage that is sufficient to provide effective therapy may be preferred in some circumstances, such as when the drug is associated with harmful side effects. Patients may generally be monitored for therapeutic or prophylactic effectiveness using assays suitable for the condition being treated or prevented, which will be familiar to those having ordinary skill in the art.

Treatment protocols for drugs, including chemotherapeutic drugs, are well known in the art, and tolerable and effective dosages and treatment frequency and duration have been established for a variety of drugs for the treatment of a large number of diseases, including various cancers. According to the invention, drugs used in combination with a conjugate of the invention may be administered according to these protocols. They may also be administered at lower or higher doses and/or durations, particularly when such treatment is determined to be safe and efficacious.

Conventional chemotherapy frequently uses a “maximum tolerated dose” (MTD) of cytotoxic (chemotherapeutic) drugs, typically every 21 days, allowing a period of rest so that healthy tissue has a chance to recover. However, recent studies also suggest a synergistic approach of lowering the dose of conventional cytotoxic agents, rescheduling their application, and combining them with agents designed to interfere with the growth pathways (signal transduction pathways), thereby effectively inhibiting the production of blood vessels. This approach, known as metronomic dosing, uses a dosing schedule as often as every day. For example, an amount as low as 25% of the MTD in combination with various signal transduction pathway inhibitors targets the endothelial cells making up the vessels and micro-vessels feeding the tumor. Endothelial cells die with much less chemo than conventional cancer cells, and the side effects to healthy tissue and the patient in general are dramatically reduced. During standard chemotherapy, the typical 21-day rest period is enough to allow the endothelial cells the chance to recover. Accordingly, combinations of the invention may be administered according to either maximum tolerated dose, metronomic dosing protocols, or any other dosing regiment or protocol known or available in the art.

In one embodiment of the invention, a combination includes a conjugate of a taxane, such as PG-TXL. Free paclitaxel (TAXOL) is associated with several undesirable side-effects. More specifically, when taxol is administered in a treatment regimen for solid tumors, patients may experience myelosuppression, i.e. bone marrow suppression that includes neutropenia, anemia, and thrombocytopenia, at elevated taxol levels. This myelosuppression is dose-limiting, and the maximum quantity of taxol that is recommended to be used in the treatment of solid tumors is 175 mg per square meter of body area every 21 days (mg/m²/21 days). Common dosages of taxol are between 135 mg/m² to about 175 mg/m² provided in a 3-hour infusion. Common side effects of taxol and standard dosages are discussed in U.S. Pat. No. 5,496,804, No. 5,641,803, and U.S. Pat. No. 5,670, 537, which are hereby incorporated by reference in their entirety. Hence, in one embodiment, the invention contemplates using a conjugate, such as PG-TXL, at any equivalent dose as taxol. However, since PG-TXL is more stable and less toxic than taxol, the invention also includes administering PG-TXL or another taxane conjugate at doses greater than typical free drug doses. In addition, PG-TXL, for example, could be administered at lower equivalent doses than those shown to be effective for taxol.

In one effort to overcome the toxic side effects associated with taxol administration, taxol is administered in combination with premedication using steroid, antihistamines, or H₂-antagonists to reduce anaphylactic reaction or other hypersensistivity responses associated with taxol treatment. Taxane conjugates may also be administered in combination with such agents, however, the invention includes administering taxane conjugates, such as PG-TXL, without pre-medication or concurrent administration of steroids, antihistamines or H₂-antagonists. When administered in combination with pre-medication with steroids, antihistamines, or H₂-antagonists, taxol may be administered at doses in excess of the previous dose-limiting amount, i.e. in excess of about 175 mg/m²/21 days. Described in U.S. Pat. No. 5,496,804.

Since taxane conjugates are less toxic than free drugs, such as TAXOL, the invention also includes administering taxane conjugates, such as PG-TXL, at doses in excess of about 175 mg/m² paclitaxel equivalents/21 days, in the absence of pre-medication or concurrent administration of steroids, antihistamines, or H₂-antagonists. Similarly, the invention also includes administering taxane conjugates, such as PG-TXL, at doses in excess of about 175 mg/m²paclitaxel equivalents/21 days, in the absence of pre-medication or concurrent administration of steroids, antihistamines, or H₂-antagonists, and in the absence of a side effect associated with the administration of taxol at the same equivalent dose, such as myelosuppression, mucositis, or peripheral neuropathy, for example. In certain embodiments, a patient is treated with a taxane conjugate, such as PG-TXL in an amount of about 200 to about 250 mg/m² paclitaxel equivalents/21 days, while in another embodiment, a patient is treated with a taxane conjugate, such as PG-TXL, in an amount in excess of about 250 mg/m₂ paclitaxel equivalents/21 days or in excess of about 275 mg/m₂ paclitaxel equivalents/21 days. The taxane conjugates may be administered at these doses in combination with other therapies or drugs, including other chemotherapeutic agents. Taxane conjugates may be administered at these doses in the presence or absence of pre-medication or concurrent administration of steroids, antihistamines, or H₂-antagonists.

In certain embodiments, drug conjugates of the invention are administered as an IV infusion. The infusion may last for any appropriate time period, which is readily determinable and assessable by one of ordinary skill in the art. For example, infusions may last for from about one to about 24 hours, although shorter or longer infusion times all fall within the scope of the invention. In specific embodiments, conjugates, including taxane conjugates such as PG-TXL, are administered as an approximately 6 hour IV infusion, while in other embodiments, the conjugates are administered as an approximately 3 hour IV infusion or an approximately 24 hour IV infusion. In certain embodiments, infusions are administered every 21 days, although more frequent and less frequent administration of conjugates is also within the scope of the invention. Appropriate time periods are known by one of skill in the art, and may be determined based upon a variety of factors, including the type of therapy or drug being used in combination with a drug conjugate. Examples of different ranges of dosage and administration schedules are provided in U.S. Pat. No. 5,670,537, which is incorporated by reference in its entirety.

In certain embodiments, drug conjugates of the invention are administered every 21 days, whereas in other embodiments, they are administered more or less frequently.

Toxicity studies, pharmacokinetics and tissue distribution of DTPA-paclitaxel have shown that in mice the LD₅₀ (50% lethal dose) of DPTA-paclitaxel observed with a single dose intravenous (iv) injection is about 110 mg/kg body weight. Direct comparison with paclitaxel is difficult to make because of the dose-volume restraints imposed by limited solubility of paclitaxel and vehicle toxicity associated with iv administration. However, in light of the present disclosure, one skilled in the art of chemotherapy would determine the effective and maximal tolerated dosages in a clinical study for use in human subjects.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

1. A therapy for the treatment of cancer comprising administering to a patient in need thereof a polymer-taxane conjugate and one or more of group comprising a camptothecin, gemcitabine, raltitrexed, cyclophosphamide, vinorelbine tartrate, flavopiridol, imatinib mesylate, epirubicin, fluorouracil, mesna, bleomycin, vinorelbine or gefitinib.

2. The therapy of claim 1, wherein the polymer of said polymer-taxane conjugate is poly-1-glutamate and the taxane of said polymer-taxane conjugate is paclitaxel.

3. The therapy of claim 2, wherein said polymer-taxane conjugate is administered at 175, 210, 225, 235 or 250 mg/m2 paclitaxel equivalents in combination with gemcitibine administered at 1250 mg/m2.

4. The therapy of claim 3, further comprising radiation therapy.

5. The therapy of claim 3, further comprising administering vinorelbine at 30 mg/m2.

6. The therapy of claim 5, further comprising radiation therapy.

7. The therapy of claim 3, further comprising administering carboplatin administered at AUC=5 or 6 or cisplatin administered at 75 or 80 mg/m2.

8. The therapy of claim 7, further comprising radiation therapy.

9. The therapy of claim 7, further comprising vinorelbine administered at 30 mg/m2.

10. The therapy of claim 9, further comprising radiation therapy.

11. The therapy of claim 2, wherein said polymer-taxane conjugate is administered at 175, 210, 225, 235 or 250 mg/m2 paclitaxel equivalents in combination with vinorelbine administered at 30 mg/m2.

12. The therapy of claim 11, further comprising radiation therapy.

13. A therapy for the treatment of cancer comprising administering to a patient in need thereof a polymer-taxane conjugate wherein said polymer-taxane conjugate is administered at 175, 210, 225, 235 or 250 mg/m2 paclitaxel equivalents in combination with carboplatin administered at AUC=5 or 6 or cisplatin administered at 75 or 80 mg/m2.

14. The therapy of claim 13, further comprising radiation therapy.

15. The therapy of claim 13, further comprising administering vinorelbine at 30 mg/m2.

16. The therapy of claim 15, further comprising radiation therapy.

17. The therapy of claim 13, wherein the polymer of said polymer-taxane conjugate is poly-1-glutamate and the taxane of said polymer-taxane conjugate is paclitaxel.

18. The therapy of claim 17, further comprising radiation therapy.

19. The therapy of claim 17, further comprising administering vinorelbine at 30 mg/m2.

20. The therapy of claim 19, further comprising radiation therapy.
